(19) United States
(12) Patent Application Publication
(10) Pub. No.: US 2014/0276718 A1
(43) Pub. Date: Sep. 18, 2014

(54) Direct Heat Ablation Catheter

(71) Applicant: Medtronic Ardian Luxembourg S.a.r.l., Luxembourg (LU)

(72) Inventors: Roman Turovskiy, San Francisco, CA (US); Robert J. Beetel, Mountain View, CA (US); Kevin M. Mauch, Windsor, CA (US); Christopher W. Storment, Sonoma, CA (US)

(73) Assignee: Medtronic Ardian Luxembourg S.a.r.l., Luxembourg (LU)

(21) Appl. No.: 14/203,933

(22) Filed: Mar. 11, 2014

Related U.S. Application Data
(60) Provisional application No. 61/789,113, filed on Mar. 15, 2013.

(51) Int. Cl. A61B 18/08

(52) U.S. Cl. CPC A61B 18/082; A61B 2018/0022
USPC 606/31

(57) ABSTRACT

Catheter apparatuses, systems, and methods for achieving renal neuromodulation by intravascular access are disclosed herein. One aspect of the present technology, for example, is directed to a treatment device including a therapeutic assembly having a central axis and a distal portion and a proximal portion axially spaced along the central axis, the therapeutic assembly including a core element extending from the proximal portion to the distal portion and a heating element disposed along at least a portion of a length of the core element. An energy source provides electrical current to the heating element to cause the heating element to increase in temperature.

### CROSS-REFERENCE TO RELATED APPLICATION(S)

This application claims the benefit of currently pending U.S. Provisional Patent Application No. 61/789,113, filed on Mar. 15, 2013, the disclosure of which is incorporated herein by reference in its entirety.

### TECHNICAL FIELD

The present disclosure relates generally to the field of tissue ablation, and some embodiments relate to the application of heat for soft tissue ablation and/or neuromodulation (e.g., renal neuromodulation).

### BACKGROUND

The sympathetic nervous system (SNS) is a primarily involuntary bodily control system typically associated with stress responses. Fibers of the SNS innervate tissue present in almost every organ system of the human body and can affect characteristics such as pupil diameter, gut motility, and urinary output. Such regulation can have adaptive utility in maintaining homeostasis or preparing the body for rapid response to environmental factors. Chronic activation of the SNS, however, is a common maladaptive response that can drive the progression of many disease states. Excessive activation of the renal SNS in particular has been identified experimentally and in humans as a likely contributor to the complex pathophysiology of hypertension, states of volume overload (such as heart failure), and progressive renal disease. For example, radiotracer dilution has demonstrated increased renal norepinephrine (“NE”) spillover rates in patients with essential hypertension.

Cardio-renal sympathetic nerve hyperactivity can be particularly pronounced in patients with heart failure. For example, an exaggerated NE overflow from the heart and kidneys of plasma is often found in these patients. Heightened SNS activation commonly characterizes both chronic and end stage renal disease. In patients with end stage renal disease, NE plasma levels above the median have been demonstrated to be predictive of cardiovascular diseases and several causes of death. This is also true for patients suffering from diabetic or contrast nephropathy. Evidence suggests that sensory afferent signals originating from diseased kidneys are major contributors to initiating and sustaining elevated central sympathetic outflow.

Sympathetic nerves innervating the kidneys terminate in the blood vessels, the juxtaglomerular apparatus, and the renal tubules. Stimulation of the renal sympathetic nerves can cause increased renin release, increased sodium (Na⁺) reabsorption, and a reduction of renal blood flow. These neural regulation components of renal function are considerably stimulated in disease states characterized by heightened sympathetic tone and likely contribute to increased blood pressure in hypertensive patients. The reduction of renal blood flow and glomerular filtration rate as a result of renal sympathetic efferent stimulation is likely a cornerstone of the loss of renal function in cardio-renal syndrome (i.e., renal dysfunction as a progressive complication of chronic heart failure). Pharmacologic strategies to thwart the consequences of renal efferent sympathetic stimulation include centrally acting sympatholytic drugs, beta blockers (intended to reduce renin release), angiotensin converting enzyme inhibitors and receptor blockers (intended to block the action of angiotensin II and aldosterone activation consequent to renin release), and diuretics (intended to counter the renal sympathetic mediated sodium and water retention). These pharmacologic strategies, however, have significant limitations including limited efficacy, compliance issues, side effects, and others. Recently, intravascular devices that reduce sympathetic nerve activity by applying an energy field to a target site in the renal artery (e.g., via radiofrequency ablation) have been shown to reduce blood pressure in patients with treatment-resistant hypertension.

### SUMMARY

The present technology is directed toward apparatus, systems and methods for tissue ablation. Particularly, some embodiments are directed toward apparatus, systems and methods for neuromodulation, including renal neuromodulation, through the direct application of heat. The heat can be applied by one or more heating elements, such as, for example, resistive heating elements, that are positioned adjacent the artery wall and heated to a desired temperature. An energy source can be included to supply electrical current to the heating element or elements.

Various embodiments provide a catheter apparatus for treatment of a human patient via renal neuromodulation. The catheter apparatus can include a therapeutic assembly having a central axis and a distal portion and a proximal portion axially spaced along the central axis. The therapeutic assembly can include a core element extending from the proximal portion to the distal portion and a heating element disposed along at least a portion of a length of the core element. In some embodiments, the core element comprises a core element and the heating element comprises a resistive element disposed on or supported by the core element. In further embodiments, the heating element may be a resistive wire wrapped around the core element. The core element can be shape set in a predetermined geometry, and it can be moveable from a delivery state to an expanded state. The expanded state can provide a geometry configured to position the heating element adjacent a vessel wall for renal neuromodulation.

In various embodiments, the resistive element can be disposed on all or substantially all of the length of the core element, or the resistive element can be disposed on or wrapped around (e.g., in the case of resistive wire) a predetermined length of the core element. A temperature sensor can be disposed adjacent (e.g., near or on the surface of) the heating element to sense temperature of the heating element. For example, in embodiments where the heating element comprises resistive element wound around the core element, the temperature sensor can be disposed adjacent the resistive element.

A processing module can be included and used to determine a temperature of the heating element based on a signal received from the temperature sensor, and to control an amount and duration of energy delivered to the heating element based on the determined temperature.

The core element can be shape set in a helical geometry and it can be configured to be delivered in a collapsed state having a first profile and transitioned to an expanded state having a second profile. For example, the first profile can be of a first diameter suitable for delivery of the core element to a renal artery and the second profile can be of a second diameter suitable to cause the heating element disposed on the core element to be placed into contact with a wall of the renal artery. In some embodiments, in the collapsed state the core element is in an elongated configuration, and in the expanded state the core element is in a helical configuration. In further embodiments, the catheter includes a second core element having a heating element disposed thereon, and the core elements can be shape set in a helical configuration.

In some embodiments, the therapeutic assembly can further comprise a body defining a lumen. The core element can be disposed within the lumen in the collapsed state and deployed from an aperture formed along a predetermined length of the body. In other embodiments, the therapeutic assembly can further comprise a support member along its central axis, and wherein in the collapsed state the core element wraps around the support member in a helical configuration such that a diameter of the therapeutic assembly is less than an inner diameter of a renal artery of an intended patient.

In still further embodiments, an apparatus for neuromodulation treatment includes a therapeutic assembly configured to be delivered to a treatment site within a vessel. The therapeutic assembly can include a heating element configured to be positioned in contact with a vessel wall to deliver heat to the vessel wall. The therapeutic assembly can also include a core element having a length, and the heating element comprises wire wound around at least a part of the length of the core element. In some embodiments, the heating element comprises wire wound around all or substantially all of the length of the core element.

In various embodiments, the therapeutic assembly includes an inflatable balloon, and the heating element can be a resistive heating element disposed on the inflatable balloon. The heating element can be a resistive element wrapped around the outer surface of the balloon, a conductive pattern printed on the surface of the balloon or a ceramic heating element disposed on a surface of the balloon. As further examples, heaters can also include other layered or laminate heating elements, which may include a metallic layer or metallic traces disposed on a flexible thin film such as a polyimide, silicone, or Teflon™ film.

A method for neuromodulation through the application of direct heat is also provided. The method can include positioning a therapeutic assembly within a blood vessel of a patient, the therapeutic assembly comprising a heating element; causing the heating element to increase in temperature thereby generating heat; and at least partially denervating tissue that is innervated by nerves located within or in proximity to the blood vessel wall via the application of the heat generated by the heating element. The method can further include positioning the therapeutic assembly within the blood vessel in a first state and expanding the therapeutic assembly to a second state such that the heating element comes into contact with an intimal surface of the wall of the blood vessel.

The therapeutic assembly can be configured to have a first diameter in the first state and a second diameter in the second state, and the first diameter can be smaller than the diameter of the inner wall of the blood vessel of the patient and the second diameter can be large enough to cause the heating element to be placed into contact with the inner wall of the blood vessel. In various embodiments, the heating element is a resistive heating element, and causing the heating element to increase in temperature comprises applying an electrical current sufficient to cause the resistive heating element to reach a predetermined temperature. The method can further include measuring a temperature of the resistive heating element during treatment and adjusting the current applied to the resistive heating element to achieve a desired temperature. Current can be applied using a current supply or a voltage supply. For example, the electrical current can be applied by applying a voltage across the resistive heating element. The voltage or potential can be applied at a level that achieves a desired temperature in the heating element.

### BRIEF DESCRIPTION OF THE DRAWINGS

The present invention, in accordance with one or more various embodiments, is described in detail with reference to the accompanying figures. The drawings are provided for purposes of illustration only and merely depict typical or example embodiments of the invention. These drawings are provided to facilitate the reader's understanding of the systems and methods described herein, and shall not be considered limiting of the breadth, scope, or applicability of the claimed invention.

FIG. 1 illustrates a system in accordance with one embodiment of the technology disclosed herein.

FIG. 2 illustrates one example of modulating renal nerves with an embodiment of the system described with reference to FIG. 1.

FIG. 3 is a diagram illustrating an example of a heating element disposed on an inflatable balloon in accordance with one embodiment of the technology described herein.

FIG. 4 is a diagram illustrating an example of a heating element having a resistive element wound around a core element in accordance with one embodiment of the technology described herein.

FIG. 5 is a diagram illustrating a catheter system using a shape-set helix as therapeutic assembly in accordance with one embodiment of the technology described herein.

FIG. 6a is a diagram illustrating an example of a therapeutic assembly in a delivery state in the renal artery in accordance with one embodiment of the technology described herein.

FIG. 6b is a diagram illustrating an example of a shape-set helix as therapeutic assembly in an expanded state in the renal artery in accordance with one embodiment of the technology described herein.

FIGS. 7a and 7b are diagrams illustrating examples of resistive heating wire wrapped around a core element in accordance with one embodiment of the technology described herein.

FIG. 8 is a diagram illustrating pitch, radius, and other metrics for a coiled heating wire wrapped about a core in accordance with one embodiment of the technology described herein.

FIGS. 9a and 9b illustrate a side view, and FIG. 9c illustrates a perspective view of a heating assembly configured to be deployed from a side opening of a catheter lumen in accordance with one embodiment of the technology described herein.

FIG. 10 illustrates a cross sectional view of the catheter and heating assembly of FIG. 9b in accordance with one embodiment of the technology described herein.

FIG. 11 is a diagram illustrating dimensions of an example helical structure having a radius, R, a pitch/2π, B, and an arcuate length, LARC, about an angle θ over an axial length, LLONG.

FIG. 12 is a diagram illustrating a representative example of connections of power and thermocouple wires to an energy source in accordance with one embodiment of the technology described herein.

FIG. 13 illustrates an example computing module that may be used in implementing various features of embodiments of the systems and methods disclosed herein.

FIG. 14 illustrates a network of nerves that make up the sympathetic nervous system, allowing the brain to communicate with the body.

FIG. 15 illustrates the kidney, innervated by the renal plexus (RP), which is intimately associated with the renal artery.

FIGS. 16a and 16b, illustrate afferent communication from the kidney to the brain and from one kidney to the other kidney (via the central nervous system).

FIG. 17a shows human arterial vasculature.

FIG. 17b shows human venous vasculature.

The figures are not intended to be exhaustive or to limit the invention to the precise form disclosed. It should be understood that the invention can be practiced with modification and alteration, and that the invention be limited only by the claims and the equivalents thereof.

### DETAILED DESCRIPTION

The present technology is generally directed to tissue ablation. More particularly, some embodiments are directed toward modulation of nerves, including nerves innervating the kidneys. Various techniques can be used to partially or completely incapacitate neural pathways, such as those innervating the kidney. The purposeful application of direct heat to tissue can induce one or more desired thermal heating effects on localized regions of the renal artery and adjacent regions of the renal plexus (RP), which lay intimately within or adjacent to the adventitia of the renal artery. The purposeful application of the thermal heating can achieve neuromodulation along all or a portion of the renal plexus (RP). Additionally direct heat could be applied for other ablative procedures, especially those requiring relatively shallow lesions. This could be useful, for example, for denervation of other nerves that lie closer to the tissue surface than the renal nerves, or for partial renal denervation.

Specific details of several embodiments of the present technology are described herein with reference to the accompanying figures. Other embodiments of the present technology can have configurations, components, or procedures different from those described herein. For example, other embodiments can include additional elements and features beyond those described herein or be without several of the elements and features shown and described herein. Generally, unless the context indicates otherwise, the terms “distal” and “proximal” within this disclosure reference a position relative to an operator or an operator's control device. For example, “proximal” can refer to a position closer to an operator or an operator's control device, and “distal” can refer to a position that is more distant from an operator or an operator's control device. The headings provided herein are for convenience only.

### I. Renal Neuromodulation

Renal neuromodulation is the partial or complete incapacitation or other effective disruption of nerves innervating the kidneys. In particular, renal neuromodulation comprises inhibiting, reducing, and/or blocking neural communication along neural fibers (i.e., efferent and/or afferent nerve fibers) innervating the kidneys. Such incapacitation can be long-term (e.g., permanent or for periods of months, years, or decades) or short-term (e.g., for periods of minutes, hours, days, or weeks). Renal neuromodulation is expected to efficaciously treat several clinical conditions characterized by increased overall sympathetic activity, and in particular conditions associated with central sympathetic overstimulation such as hypertension, heart failure, acute myocardial infarction, metabolic syndrome, insulin resistance, diabetes, left ventricular hypertrophy, chronic and end stage renal disease, inappropriate fluid retention in heart failure, cardio-renal syndrome, osteoporosis and sudden death. The reduction of afferent neural signals contributes to the systemic reduction of sympathetic tone/drive, and renal neuromodulation is expected to be useful in treating several conditions associated with systemic sympathetic overactivity or hyperactivity. Renal neuromodulation can potentially benefit a variety of organs and bodily structures innervated by sympathetic nerves. For example, a reduction in central sympathetic drive may reduce insulin resistance that afflicts patients with metabolic syndrome and Type II diabetics.

### II. Selected Embodiments of Treatment Systems

FIG. 1 illustrates a system 1 in accordance with an embodiment of the present technology. The system 1 includes a renal neuromodulation system 10 (“system 10”). The system 10 includes an intravascular or intraluminal treatment device 12 that is operably coupled to an energy source or console 26. Energy source or console 26 can include, for example, a current or voltage source to power direct heat heating elements. In other embodiments (e.g., in hybrid applications using multiple energy modalities) energy source or console 26 can include an RF energy generator, a cryotherapy console, an ultrasonic signal generator or other energy source. One advantage that may be obtained by using direct heat thermal ablation over other energy modalities is that with direct heat, the energy source may be simplified. Rather than using a relatively complex RF generator, for example, the direct heat embodiments can be implemented with a simplified AC or DC power source. For example, a DC power supply configured to convert AC power to a DC voltage can be used to power the heating elements. Because heating elements can configured for low power consumption, in some embodiments a relatively inexpensive AC to DC power supply can be used that can derive its power directly from a conventional AC mains wall outlet. Accordingly, the energy generator for direct heat embodiments can be less complex and lower cost as compared to generators used for other modalities (e.g., RF modalities requiring more complex RF signal generators). Indeed, in other applications, the generator can comprise one or more batteries to power the heating elements. Accordingly, although illustrated as a separate console in FIG. 1, the energy source used to power the direct heat heating elements could be housed in handle 34, or otherwise configured into a lower-cost, smaller, portable or hand-held package.

In the embodiment shown in FIG.1, the treatment device 12 (e.g., a catheter) includes an elongated shaft 16 having a proximal portion 18, a handle 34 at a proximal region of the proximal portion 18, and a distal portion 20 extending distally relative to the proximal portion 18. The treatment device 12 further includes a therapeutic assembly or treatment section 21 at the distal portion 20 of the shaft 16. The therapeutic assembly 21 can include a neuromodulation assembly, which can comprise actuators such as one or more electrodes or energy delivery elements. In one embodiment, direct heat elements are used exclusively for treatment. In other embodiments, other energy delivery elements for other treatment modalities (e.g., RF emitters, ultrasonic transducers, a cryotherapeutic cooling assembly, etc.) can be included with the direct heat heating elements.

Upon delivery to the target treatment site within the renal blood vessel, the therapeutic assembly 21 is further configured to be deployed into a treatment state or arrangement for delivering energy at the treatment site and providing therapeutically effective electrically induced and/or thermally induced renal neuromodulation. In some embodiments, the therapeutic assembly 21 may be placed or transformed into the deployed state or arrangement via remote actuation, e.g., via an actuator 36, such as a knob, button, pin, or lever carried by the handle 34. In other embodiments, however, the therapeutic assembly 21 may be transformed between the delivery and deployed states using other suitable mechanisms or techniques.

The proximal end of the therapeutic assembly 21 is carried by or affixed to the distal portion 20 of the elongated shaft 16. A distal end of the therapeutic assembly 21 may terminate with, for example, an atraumatic rounded tip or cap. Alternatively, the distal end of the therapeutic assembly 21 may be configured to engage another element of the system 10 or treatment device 12.

For example, the distal end of the therapeutic assembly 21 may define a passageway for engaging a guide wire (not shown) for delivery of the treatment device using over-the-wire (“OTW”) or rapid exchange (“RX”) techniques.

The energy source or console 26 is configured to generate a selected form and magnitude of energy for delivery to the target treatment site via the therapeutic assembly 21. A control mechanism, such as foot pedal 32 or other operator control, may be connected (e.g., pneumatically connected or electrically connected) to the console to allow the operator to initiate, terminate and, optionally, adjust various operational characteristics of the energy generator, including, but not limited to, power delivery.

The system 10 may also include a remote control device (not shown) that can be positioned in a sterile field and operably coupled to the therapeutic assembly 21. The remote control device can be configured to allow for selective activation of the therapeutic assembly 21. For example, the remote control device can be configured to allow the operator to initiate, terminate and, optionally, adjust various operational characteristics of the energy generator. In some embodiments, a control mechanism (not shown) may be built into the handle assembly 34 allowing operator control through actuation of buttons, switches or other mechanisms on the handle assembly 34.

The energy source 26 can be configured to deliver the treatment energy under the control of an automated control algorithm 30, under the control of the clinician, or via a combination thereof. In addition, the energy source or console 26 may include one or more evaluation or feedback algorithms 31 that can be configured to accept information and provide feedback to the clinician before, during, and/or after therapy (e.g., neuromodulation). Feedback can be provided in the form of audible, visual or haptic feedback. The feedback can be based on output from a monitoring system (not shown). The monitoring system can be a system including sensors or other monitoring devices integrated with treatment device 12, sensors or other monitoring devices separate from treatment device 12, or a combination thereof. The monitoring devices of the monitoring system can be configured to measure conditions at the treatment site (e.g., the temperature of the tissue being treated), systemic conditions (e.g., patient vital signs), or other conditions germane to the treatment or to the health and safety of the patient.

The energy source 26 can further include a device or monitor that may include processing circuitry, such as one or more microprocessors, and a display 33. The processing circuitry may be configured to execute stored instructions relating to the control algorithm 30. The energy source 26 may be configured to communicate with the treatment device 12 (e.g., via the cable 28) to control the neuromodulation assembly and/or to send signals to or receive signals from the monitoring system. The display 33 may be configured to provide indications of power levels or sensor data, such as audio, visual or other indications, or may be configured to communicate the information to another device. For example, the console 26 may also be operably coupled to a catheter lab screen or system for displaying treatment information (e.g., nerve activity before and after treatment, effects of ablation, efficacy of ablation of nerve tissue, lesion location, lesion size, etc.).

The energy source or console 26 can be configured to control, monitor, supply, or otherwise support operation of the treatment device 12. In other embodiments, the treatment device 12 can be self-contained and/or otherwise configured for operation without connection to the energy source or console 26. As shown in the example of FIG. 1, the energy source or console 26 can include a primary housing having a display 33.

In some embodiments, the energy source or console 26 can include a processing device or module (not shown) having processing circuitry, e.g., a microprocessor. The processing device can be configured to execute stored instructions relating to the control algorithm 30, the evaluation/feedback algorithm 31 and other functions of the device. Furthermore, the energy source or console 26 can be configured to communicate with the treatment device 12, e.g., via the cable 28. For example, the therapeutic assembly 21 of the treatment device 12 can include a sensor (not shown) (e.g., a recording electrode, a temperature sensor, a pressure sensor, or a flow rate sensor) and a sensor lead (not shown) (e.g., an electrical lead or a pressure lead) configured to carry a signal from the sensor to the handle 34. The cable 28 can be configured to carry the signal from the handle 34 to the energy source or console 26.

The energy source or console 26 can have different configurations depending on the treatment modality of the treatment device 12. For example, energy source or console 26 can be configured with an energy source configured to provide the source of power to energize the direct heat heating elements. As noted above, in some embodiments, the energy source can be implemented in handle 34. One or more batteries (e.g., rechargeable or disposable) can be integrated into handle 34 along with a processing module or other control software and electronics to control power delivery to the heating elements. Such a control module can be configured to control power delivery based on factors such as treatment times, temperature ranges and thresholds, current ranges and thresholds, tissue impedance measurements and the like. In other embodiments, the energy source or console 26 can have other suitable configurations.

FIG. 2 illustrates one example of modulating renal nerves with an embodiment of the system 10. In this embodiment, the treatment device 12 provides access to the renal plexus (RP) through an intravascular path (P), such as a percutaneous access site in the femoral (illustrated), brachial, radial, or axillary artery to a targeted treatment site within a respective renal artery (RA). As illustrated, a section of the proximal portion 18 of the shaft 16 is exposed externally of the patient. By manipulating the proximal portion 18 of the shaft 16 from outside the intravascular path (P), the clinician may advance the shaft 16 through the sometimes tortuous intravascular path (P) and remotely manipulate the distal portion 20 of the shaft 16. Image guidance, e.g., computed tomography (CT), fluoroscopy, intravascular ultrasound (IVUS), optical coherence tomography (OCT), or another suitable guidance modality, or combinations thereof, may be used to aid the clinician's manipulation. Further, in some embodiments, image guidance components (e.g., IVUS, OCT) may be incorporated into the treatment device 12 itself.

After the therapeutic assembly 21 is adequately positioned in the renal artery (RA), it can be radially expanded, inflated or otherwise deployed using the handle 34 or other suitable means until the neuromodulation assembly is positioned at its target site and the nerve-monitoring device is in stable contact with the inner wall of the renal artery (RA). The purposeful application of energy from the neuromodulation assembly is then applied to tissue to induce one or more desired neuromodulating effects on localized regions of the renal artery and adjacent regions of the renal plexus (RP), which lay intimately within, adjacent to, or in close proximity to the adventitia of the renal artery (RA). The purposeful application of the energy may achieve neuromodulation along all or at least a portion of the renal plexus (RP).

In various embodiments, therapeutic assembly 21 can include one or more heat actuators or heating elements configured to directly apply heat at the treatment location to accomplish the desired neuromodulation effect. Particularly, in some embodiments, the application of heat from therapeutic assembly 21 can be made directly to the tissue to accomplish the desired treatment. Even more particularly, in such embodiments, direct heat can be applied directly to the vessel wall and the heat can transfer by conduction to the depth of the renal plexus (RP), which lay intimately within, adjacent to, or in close proximity to the adventitia of the renal artery (RA). This can be accomplished by the positioning of a heating element along the arterial wall and applying heat by way of the heating element. Direct heat modalities can be distinguished from indirect heat modalities based on the manner in which heat is applied to the vessel wall. With direct heat applications, or direct heating elements, the tissue is heated by thermal transfer of heat produced by the heating element. Particularly, the heating element is placed in contact with (or in close proximity to) the tissue and is heated to an elevated temperature. The thermal energy or heat from the heated element is transferred to the tissue, thereby heating the tissue. This is in contrast to, for example, an RF application in which RF energy propagated from a first electrode to a second electrode generates heat in the tissue.

One or more heating elements can be included with and deployed by therapeutic assembly 21 in a number of different configurations. In various embodiments, heating elements can be resistive heating elements configured to generate heat in response to an electric current flowing through a resistive wire or other resistive element. Examples of resistive elements can include a coil, ribbon, or strip of wire made from resistive material that generates heat due to the resistance to the flow of current from a power source. Examples of resistive wire suitable for such applications are Nichrome 80 and Nichrome 60, stainless steel and constantan, although other resistive wire can be used. Although numerous embodiments are described herein using resistive wire, one of ordinary skill in the art will appreciate that other resistive components can be used to form the heating elements.

The heating elements can be disposed on or with any of a number of different deployment mechanisms to allow positioning of one or more heating elements adjacent the vessel wall for treatment. The delivery structure can be an expandable or deflectable structure that is used to position the heating element for deployment at the vessel wall. The delivery structure and heating elements can have a collapsed or other low-profile geometry for suitable for delivery to the treatment site, and an expanded geometry that allows the heating elements to be brought into contact with the vessel wall for treatment.

One example of such a deployment mechanism is an inflatable balloon. FIG. 3 is a diagram illustrating an example of a heating element disposed on an inflatable balloon in accordance with one embodiment of the technology described herein. With reference to FIG. 3, therapeutic assembly 21 includes an inflatable balloon 41, an atraumatic flexible tip 42, and a heating element 43. In this example, heating element 43 is disposed in a helical configuration about the surface of inflatable balloon 41. In operation, inflatable balloon 41 and heating element 43 are delivered to the treatment site in a folded, collapsed, shrunk, or otherwise undeployed configuration. At the treatment site, the inflatable balloon 41 is inflated, causing heating element 43 to contact the vessel walls. Although one heating element 43 coil is illustrated, multiple coils can be included on inflatable balloon 41.

In one embodiment, heating element 43 is a resistive metallic strip printed on inflatable balloon 41. Printing techniques can be used to print one or more conductive ink patterns on the inflatable balloon. For example, conductive polymers, metalized inks or other conductive inks can be printed onto the inflatable balloon 41 to create heating element 43 in the desired pattern. Conductive inks can use a blend of conductive material (e.g., silver or copper) and carbon in a polymer resin to achieve the desired resistance for an intended printing geometry. Viscosities of the inks can be chosen to achieve desired flow properties and pattern geometries for the balloon application. Examples of conductive inks are those inks available from DuPont Microcircuit Materials.

Alternatively, a wire heating element can be wound around inflatable balloon 41 and put into contact with the vessel wall when inflatable balloon 41 is expanded. In one embodiment, a wire is wrapped back upon itself and twisted to form a flexible braid, which can be wound around the balloon 41. Rounded, flat or even ribbon wire can be used for heating element 43. The wires or elements used for heating element 43 can be insulated to avoid shorting and to avoid electrical contact with the vessel wall. Additionally the wires or elements used for heating element 43 can be disposed internal to the balloon.

As yet another embodiment, a shape-set core element can be used to define an expanded configuration of the heating element 43. For example, the heating element 43 can comprise a coil of heating wire wound around a shape-set core. As a further example, the heating element 43 can comprise a coil of Nichrome wire wound around a Nitinol core. The Nitinol core can be shape set to the final expanded shape so that the heating element 43 takes this shape upon deployment. FIG. 4 is a diagram illustrating an example of a heating element 43 having a resistive wire 44 wound around a core element 46. In this example, resistive heating wire 44 (e.g., Nichrome wire) is wound around a core element 46 (e.g., shape set Nitinol). Core element 46 can be a shape set core. Although illustrated as straight for ease of illustration and clarity, the shape-set core element 46 can be shape set in a desired expanded configuration. For example, core element 46 can be shape set to a helical or spiral configuration such that upon deployment, heating assembly 43 forms a helical shape such as that shown in FIG. 3.

Also shown in FIG. 4, heating wire 44 is insulated wire having a conductive core 44b surrounding by an insulating layer 44a. Additionally an insulating sheath 45 can be provided to avoid electrical contact with the vessel wall. The pitch of heating wire 44 can be adjusted to control the amount of wire and heat applied to the vessel wall. The pitch can be uniform or it can vary along the length of the heating element. The pitch of heating wire 44 affects the concentration of heat generated by heating element 43 and the flexibility of the heating element 43. For example, a lower pitch (tighter spacing) can provide a higher concentration of heat along the element. The pitch of heating wire 44 can range, in some embodiments, from 2 mm-25 mm, although other pitches above and below this range can be used. The relationship between pitch, wire length and assembly length are discussed below with reference to FIG. 8.

Shape setting of Nitinol, which is a well-known technique in the art, can be accomplished for this and other embodiments by forming and then constraining the Nitinol into the desired shape, and then heat-treating the material while it is held in the desired shape. Heat can be applied to adjust the Austenite Finish (A_{f}) temperature (sometimes referred to as reverse-transformation finish temperature) of the Nitinol. In some embodiments, the A_{f} is set at or near the operating temperature of the heating element during treatment. Utilizing Nitinol with an A_{f} just below the desired operating temperature range can ensure that the heating element does not open up to its expanded configuration until after it is positioned at the treatment site and current applied to heat the heating elements. The A_{f} temperature of the Nitinol core may be set around body temperature to make sure NiTi is in its superelastic state right after it is introduced into the body or above body temperature but below the active (ablation) temperature of the main heating element. The latter embodiment allows the overall structure to be softer and more flexible until the ablation sequence is initiated.

Another embodiment uses a ceramic micro heater attached to the surface of an inflatable balloon 41 using, for example, adhesives. For example, one embodiment uses heater part number 343-HEATER-2X10 (available from Allectra GmbH, Traubeneichenstr. 62-66; D-16567 Schönfliess b. Berlin, Germany) to form heating elements 43. As would be apparent to one of ordinary skill in the art after reading this description, similar heating elements of different sizes and configurations can be used. In various embodiments, thermally conductive material can be applied to the outer surface of the heater to aid in transfer of heat from the heating element to the vessel wall.

Instead of using a balloon as a deployment mechanism, other expanding or self-expanding devices can be used to place the heating element against the vessel wall for the application of direct heat to the treatment site. For example, an expanding stent or Nitinol “cage” could be used to place the heater(s) in contact with the tissue. As another example, an expandable structure such as a shape set Nitinol helix can be used to place the heating element into contact with the vessel wall. These solutions allow positioning of the electrodes without occluding renal blood flow during the ablation procedure. Because blood flow past the electrodes may transfer heat away from the direct heat elements, power delivery can be adjusted accordingly to maintain desired temperatures.

FIG. 5 is a diagram illustrating a catheter system using a shape-set helix as therapeutic assembly 21 in accordance with one embodiment of the technology described herein. A heating assembly 48 is provided at the distal end of catheter shaft 16. In this example, heating assembly 48 is configured to be expanded in a helical or spiral configuration such that, when expanded, part or all of the helix contacts the vessel wall to deliver heat. Although a single helix is shown, double- and multi-helix configurations can be used as well.

In some embodiments, heating assembly 48 comprises a resistive heating element disposed on a core element. For example, heating assembly 48 can include heating wire wound around a shape-set core element. As explained in greater detail below, the heating wire can be a resistive wire having resistance chosen such that the heating element heats to a desired temperature upon the application of a given amount of current. The shape set core can be made from Nitinol or other suitable material.

In the embodiment of FIG. 5 (and other embodiments described herein), a balloon is not used to expand the heating assembly 48 from its delivery to its expanded state. Instead, the heating assembly 48 (e.g., the shape-set core and the resistive wire) are expanded by control mechanisms at the proximal end of treatment device 12. The transformation may be initiated using an arrangement of device components as described herein with respect to the particular embodiments and their various modes of deployment. In accordance with one or more embodiments of the present technology, the therapeutic assembly may be deployed by a control member. The control member may be, for example, a pull or tension wire, a guide wire, a shaft or stylet engaged internally or externally with the support structure of the therapeutic assembly to apply a deforming or shaping force to the therapeutic assembly 21 to transform it into its expanded state. Further, the modality used to transform the therapeutic assembly 21 from the delivery state into the expanded state may, in various embodiments, be reversed to transform the therapeutic assembly 21 back to the delivery state from the expanded state.

For example, operation of actuator 36 on treatment device 12 can be used to push support member 23 distally from the proximal end. Where the distal tip of heating assembly 48 is fixed to the distal end of support member 23, this action increases the length over which heating assembly 48 is wound, causing it to collapse onto support member 23. Likewise, when support member 23 is retracted, this decreases the length over which the helix is wound, causing it to expand radially. As another example, a helical heating assembly 48 can be contained within a sheath and operation of actuator 36 retracts the sheath to deploy heating assembly 48. This exposes heating assembly 48, allowing it to expand. To collapse heating assembly 48, the sheath can be extended back over the helix. Examples of helical assemblies and deployment mechanisms are described in more detail in U.S. patent application Ser. No. 13/281,360, filed Oct. 25, 2011; U.S. patent application Ser. No. 13/281,361, filed Oct. 25, 2011; and U.S. patent application Ser. No. 13/281,395, filed Oct. 25, 2011, each of which are incorporated by reference herein in their entirety.

FIG. 6a is a diagram illustrating an example of a therapeutic assembly 21 in a pre-expanded delivery state in the renal artery RA. Therapeutic assembly 21 in this example comprises a heating assembly 48 configured as a helical element wrapped about a support structure 23. FIG. 6b is a diagram illustrating an example of a shape-set helix as therapeutic assembly 21 in an expanded state in the renal artery RA. As shown in FIG. 6a, in the delivery state, heating assembly 48 is wrapped around the support member 23 in a relatively tight configuration to allow clearance between therapeutic assembly 21 and the vessel walls.

The collapsed or delivery arrangement of the therapeutic assembly 21 defines a low profile about the longitudinal axis of the assembly such that a transverse dimension of the therapeutic assembly 21 is sufficiently small to define a clearance distance between an arterial wall 55 and the treatment device 12. The delivery state facilitates insertion and/or removal of the treatment device 12 and, if desired, repositioning of the therapeutic assembly 21 within the renal artery RA.

In the collapsed configuration where the helical heating elements 22 are in contact with the inner support member 23 of the therapeutic assemble, for example, the geometry of inner support member 23 facilitates movement of the therapeutic assembly 21 through a guide catheter to the treatment site in the renal artery RA. Moreover, in the collapsed configuration, the therapeutic assembly 21 is sized and shaped to fit within the renal artery RA and has a diameter that is less than a renal artery inner diameter and a length (from a proximal end of the therapeutic assembly 21 to a distal end of the therapeutic assembly 21) that is less than a renal artery length. Furthermore, as described in greater detail below, the geometry of the support structure is arranged to define (in the delivery state) a minimum transverse dimension about its central axis that is less than the renal artery inner diameter 52 and a maximum length in the direction of the central axis that is preferably less than the renal artery length 54. In one embodiment, the minimum diameter of the therapeutic assembly 21 is approximately equal to the interior diameter of the elongated shaft 16.

The distal portion 20 of the shaft 16 may flex in a substantial fashion to gain entrance into a respective renal artery by following a path defined by a guide catheter, a guide wire, or a sheath. For example, the flexing of distal portion 20 may be imparted by the guide catheter, such as a renal guide catheter with a preformed bend near the distal end that directs the shaft 16 along a desired path from the percutaneous insertion site to the renal artery RA. In another embodiment, the treatment device 12 may be directed to the treatment site within the renal artery RA by engaging and tracking a guide wire (e.g., guide wire 66 of FIG. 2) that is inserted into the renal artery RA and extends to the percutaneous access site. In operation, the guide wire preferably is delivered first into the renal artery RA and the elongated shaft 16 comprising a guide wire lumen is then passed over the guide wire into the renal artery RA. In some guide wire procedures, a tubular delivery sheath may be passed over the guide wire (i.e., the lumen defined by the delivery sheath slides over the guide wire) into the renal artery RA. Once the delivery sheath is placed in the renal artery RA, the guide wire may be removed and exchanged for a treatment catheter (e.g., treatment device 12) that may be delivered through the delivery sheath into the renal artery RA.

Furthermore, in some embodiments, the distal portion 20 can be directed or “steered” into the renal artery RA via the handle assembly 34 (FIGS. 1 and 2), for example, by an actuatable element 36 or by another control element. Alternatively, or in addition, the treatment device 12 and its distal portion 20 may be flexed by being inserted through a steerable guide catheter (not shown) that includes a preformed or steerable bend near its distal end that can be adjusted or re-shaped by manipulation from the proximal end of the guide catheter.

The maximum outer dimensions (e.g., diameter) of any section of the elongated shaft 16 and the therapeutic assembly 21 in a collapsed delivery configuration can be defined by an inner diameter of the guide catheter through which they are passed. In one particular embodiment, for example, an 8 French guide catheter having, for example, an inner diameter of approximately 0.091 inch (2.31 mm) may be used as a guide catheter to access the renal artery. Allowing for a reasonable clearance tolerance between helical heating assembly 48 and the guide catheter, the maximum outer dimension of the therapeutic assembly 21 is generally less than or equal to approximately 0.085 inch (2.16 mm). As further examples, a 6 French guide catheter with an inner diameter=0.070″, and an outer diameter=0.082″ can be used as can a 5 French guide catheter with an inner diameter=0.053″, and an outer diameter=0.067″. Catheters with other inner diameter dimensions can also be used and the maximum outer dimensions of therapeutic assembly can be specified accordingly.

After locating the therapeutic assembly 21 in the renal artery RA, the therapeutic assembly 21 is transformed from its delivery state (collapsed as shown in FIG. 6a) to its expanded state or arrangement (expanded as shown in FIG. 6b). Particularly, the helical heating element 22 is expanded such that heating element 22 contacts artery wall 55. Energy may be delivered to heating elements surrounding the preformed core (e.g., resistive wire wrapped around the core) and a helical lesion is formed.

Further manipulation of the helical heating element within the respective renal artery RA establishes apposition of the therapeutic assembly 21 against the tissue along an interior wall 55 of the respective renal artery RA. For example, as shown in FIGS. 6a and 6b, the therapeutic assembly 21 is expanded within the renal artery RA such that the helical heating element 48 is in contact with the renal artery wall 55. Note that although only one heating element 48 is shown, multiple heating elements 48 can be included.

In some embodiments, manipulation of the proximal portion may facilitate contact between the helical heating assembly 48 and the wall 55 of the renal artery RA. The helical heating assembly 48 is operated such that the contact force between the renal artery inner wall 55 and the helical heating assembly 48 does not exceed a maximum value. For example, the deployment mechanism may comprise a gauge to ensure that the contact force between the therapeutic assembly 21 and the artery wall 55 is less than a predetermined value for arteries of different sizes. In addition, the helical heating assembly 48 and the distal support section 23 may provide for a consistent contact force against the arterial wall 55 that may allow for consistent lesion formation. Further, electrodes, pressure sensors, or other sensors can be included to allow impedance measurements to be made to detect contact with the vessel wall.

In other embodiments, the heating elements are not continuous about the length of heating assembly 48. For example, thermal insulation can be provided at one or more locations along the length of heating assembly 48 to create a set of discrete points where thermal energy is restricted from transferring to the tissue. Likewise, discrete heating elements can be provided at locations along the length of heating assembly 48 so that thermal energy is transferred at discrete points along the assembly. In this manner, rather than creating a continuous lesion, lesions can be created in a helical pattern. In further embodiments, thermal insulation can be provided on the inner surface of the helical assembly to minimize the amount of heat transferred to the blood. This can allow for a more efficient operation of the device.

As described above with reference to FIG. 5, and similar to the embodiment described above with reference to FIG. 4, heating assembly 48 can comprise heating elements disposed on a core element. The core element can be, for example, a Nitinol core, shape set in a helical pattern. In other embodiments, core element is not a shape set element. FIGS. 7a and 7b are diagrams illustrating examples of resistive heating wire wrapped around a core element. The examples shown in FIGS. 7a and 7b show only a segment of a core wrapped with resistive heating wire. As would be apparent to one of ordinary skill in the art after reading this description, resistive heating wire can be wrapped about the entire helical core, or about parts of the helical core at which the application of heat is desired.

As shown in FIGS. 7a and 7b a core element 57 is provided. As described above, core element 57 can be a Nitinol core or other like core, shape set into the desired expanded geometry. Resistive heating wire 58 is wrapped about the core 57 at the desired pitch. As shown, resistive heating wire 58 is electrically insulated wire. While the insulation can provide electrical isolation, thermally conductive insulation materials can be used to allow good conduction of heat. Polyimide is an example of thermally conductive insulation, although other insulating materials can be used.

Resistive heating wire 58 can be any conductive wire with the desired conductivity/resistance to provide the desired level of heating with applied AC, DC or RF energy. The resistance of the wire can be chosen such that the heating element reaches a desired temperature at a given level of applied current. In one embodiment, resistive heating wire 58 is Nichrome 80 or Nichrome 60 wire, although other wire can be used. Nichrome 80 wire, is resistive wire with 80% Nickel, 20% Chromium. Nichrome 60 wire, is resistive wire with 60% Nickel, 40% Chromium. The composition, length and diameter of the wire affect its resistance and heating characteristics. In some embodiments, the wire chosen is a small gauge wire having a resistance of approximately 80 Ohms, although other resistance values can be used. Desired voltage/current requirements are a consideration when choosing heating element resistance. For example, in some embodiments, resistance is selected such that desired heating characteristics can be achieved using off-the-shelf batteries or small-form-factor rechargeable batteries as a power supply.

An outer sheath 59 can be provided as well to encase heating elements 48 as well as wires used in the therapeutic assembly 21. For example, the wires can include wires used to deliver current to the resistive wire 58 for heat generation and thermocouple wires connected to a thermocouple or other temperature sensor to provide temperature feedback to console 26. Outer sheath 59 can also be made of thermally conductive material so as not to inhibit thermal transfer from heating assembly 48 to the vessel wall.

The embodiment shown in FIG. 7a is wrapped at a looser pitch than the example in FIG. 7b. The pitch, for a given core/wire combination, affects concentration of heat and flexibility of the assembly. The resistive wire in these embodiments is shown in a single coil configuration. Double helix and multiple-coil configurations can be implemented, as can braided wire configurations. Additionally, the coil can be configured to double or wrap back on itself, so that the endpoints of the coil are at or near the proximal end of the therapeutic assembly. This can facilitate electrical connection to wires connecting the coil back to the energy source or console 26. A double- or multi-helix configuration can also allow greater coverage in a shorter length.

In various embodiments, the entire length of core element 57 can be wrapped with resistive wire to form a continuous electrode. Where core element 57 is in a helical configuration, wrapping the entire length (or substantially all the length, or even most of the length) provides a helical heating element. In other embodiments, only portions of core element 57 are wrapped to create discrete heating elements along the length of core element 57. The lengths of the discrete heating elements can be predefined, as can their positions along the core element 57. In some embodiments, discrete heating elements along core element 57 can be individually connected to an energy source, while in other embodiments they can be connected together. For example, discrete heating elements along core element 57 can be connected together in series with one another.

FIG. 8 is a diagram illustrating pitch, radius, and other metrics for a coiled heating wire wrapped about a core. The radius, R, is the maximum distance, center to center, of the wire across the core. The pitch, P, the distance, center to center, from one turn of wire to the corresponding point on an immediately adjacent turn.

The length of one turn of wire is given by:

$$LengthT = 2 π \sqrt{{radius}^{2} + {( \frac{pitch}{2 π} )}^{2}} \qquad ( A )$$

The number of turns in the heating element is given by the length of the heating element divided by the pitch of the coil.

# Turns=Length/Pitch (B)

The overall length of the heating wire is given by the length of each turn, times the number of turns.

Length of heating wire=LengthT*# Turns (C)

The overall resistance of the length of wire is given by

Resistance of wire=Ω/ft*Length of heating wire (ft) (D)

FIGS. 9a, 9b, 9c and 10 illustrate a side-deployed shape set heating assembly 48 in accordance with one embodiment of the technology described herein. In some embodiments, assembly 48 includes a resistive wire heating element 58 wound around a core element 57. FIGS. 9a and 9b illustrate a side view, and FIG. 9c illustrates a perspective view of assembly 48 configured to be deployed from a side opening, or aperture, at the distal end 20 of a catheter shaft 16. In the example illustrated in FIG. 9a the length of the therapeutic assembly 21 L_{LONG} is fixed, and heating assembly 48 is disposed in an elongated configuration within the catheter lumen. Although heating assembly 48 may not in all cases be perfectly linear, it can be sufficiently linear to be contained within the radius defined by the catheter body.

Because the length of the therapeutic assembly 21 L_{LONG} is fixed, feeding the heating assembly 48 in the distal direction causes a greater length of heating assembly 48 to be contained within the fixed axial length L_{LONG}, causing the radius of the heating assembly 48 to expand. This is shown in FIGS. 9b and 9C, in which the heating assembly 48 is fed or pushed toward the distal end. This causes a greater length of heating assembly 48 to be pushed into the fixed length L_{LONG}, resulting in heating assembly moving outwardly from the lumen. Where heating assembly 48 includes a shape-set core, it can expand into the shape set geometry. For example, heating assembly 48 can be expanded into a helical configuration. FIG. 10 illustrates an axial view of FIGS. 9B and 9C, showing heating assembly 48 expanded radially about an axis parallel to catheter shaft 23.

FIG. 11 is a diagram illustrating dimensions of an example helical structure having a radius, R, a pitch/2π, B, and an arcuate length, L_{ARC}, about an angle θ over an axial length, L_{LONG}. FIG. 10 can be used to describe how decreasing the length L_{LONG} with a fixed length of heating wire can increase the radius or number of turns, or both, of the heating element. The total length of the heating element, L_{ARC}, is given by

L_{ARC}=θ\sqrt{R²+B²}

As indicated above, one way to expand the shape-set helix is to feed more of the heating element into a given length L_{LONG} of the therapeutic assembly 21. Feeding a longer length of wire into a given length L_{LONG} of therapeutic assembly 21 results in a greater radius of wire, a greater pitch or both. When the heating element is shape set into a larger radius helix, feeding the wire into the fixed-length therapeutic assembly 21 allows the heating element to take its desired shape. Likewise, shortening the length L_{LONG} of the therapeutic assembly while keeping the length of the heating element L_{ARC} constant yields the same effect.

One example of a heating element that can be expanded to a helical shape that contacts the vessel wall at 75% circumferential coverage has an overall length of 19.5 mm, and would have a longitudinal dimension L_{LONG} of 10 mm long in an 8 mm vessel, 15 mm long in a 6 mm vessel, 17.6 mm long in a 4 mm vessel. An example of this heating element can be made by coiling a resistive wire around a core or mandrel. This can use, for example, a 30 AWG Nichrome 80 wire (0D=0.010″) with 0.002″ Polyimide insulation coiled around a 0.012″ core with a coil gap of 0.005″ between turns. For a heating element length of 19.5 mm, the length of wire would be 5.4 inches.

Various examples described above use a helical heating element for delivery of heat treatment to the vessel wall. Single, double or multi-helix configurations can be provided, and the helixes can be self expanding to aid in deployment. Radiopaque markers can be provided to aid in visualization and placement. For example, radiopaque markers can be disposed at the beginning and the end of the heating element to allow identification and placement of the end points of the heating element.

One challenge with a direct heat modality for neuromodulation is the cooling effect of the blood flow on the heating elements. If convection removes too much heat from the heating element at the surface of the vessel, insufficient heat will be conducted through the vessel wall to the target nerves. Embodiments involving occlusion of blood flow, such as balloon-deployed embodiments, can avoid this effect by preventing blood from flowing past the heating elements. Such embodiments can include, for example, embodiments where one or more heating elements are on the surface of the balloon. Such embodiments can also include configurations where one or more heating elements are located at the end of the balloon, in which case the element may be exposed to standing blood.

Embodiments in which blood flow is not occluded can use other techniques to reduce or minimize the cooling effect of the blood on the heating elements. For example, in some embodiments the inner surface of the heating elements (the surface or surfaces not contacting the vessel wall during treatment) can be provided with thermal insulation to reduce heat transfer from the heating element to the blood. Silicone or other thermal insulating material can be used in the sheath or as a separate insulating layer. Of course, the heat loss can also be compensated for by increasing power delivered to the heating elements.

FIG. 12 is a diagram illustrating a representative example of connections of power and thermocouple wires to an energy source or console 26, in accordance with one embodiment of the technology described herein. As shown in the example of FIG. 12, the coil of heating wire 58 (e.g., Nichrome 60 or Nichrome 80) is electrically connected at one end to a first terminal of an energy source 26, and at the other end to a second terminal of the energy source. AC, DC or RF energy is sent through heating wire 58 at sufficient levels to cause heating of heating assembly 48 to the desired temperature.

A thermocouple TC, thermistor, or other temperature sensor can be provided and is illustrated in the example of FIG. 12 as being disposed at or near the center of the active portion of heating assembly 48. Multiple temperature sensors can be used to sense temperature at different points along heating assembly 48. A temperature sensor in or on surface of heating element or on the tissue surface can be used to inform the control of energy delivery as described below.

Thermocouple TC (or other temperature sensor(s)) is electrically connected to console 26 at a, b to provide temperature feedback. Such feedback can be used, for example, to check the temperature of heating assembly 48 during treatment. Accordingly, the delivery of heat to the treatment site can be controlled based on sensed temperature. The delivery of heat can be controlled by controlling the voltage levels provided by the energy generator. In other embodiments, current or power control can be used to limit the amount of energy delivered to the heating element, and thus its maximum achievable temperature. For temperature control, a temperature set point can be predetermined and programmed into the system (e.g., using control algorithm 30 or evaluation/feedback algorithms 31). The system can be programmed to maintain the heating element temperature at the desired set point during treatment. LEDs in the handle, screen displays on console 26 or other mechanisms can be used to indicate when the one or more heating elements are at the desired operating temperature. Safeguards can be built into the system to prevent the heating element(s) from going above a set maximum temperature. Such safeguards (e.g., using control algorithm 30 or evaluation/feedback algorithms 31) can be programmed to measure the temperature of the heating element(s) and cut or reduce power to the system when a maximum temperature level is achieved. Warnings or messaging can be used to advise the operator of operating temperatures and temperature conditions over or under the desired operating range.

Thermal effects can include both thermal ablation and non-ablative thermal alteration or damage (e.g., via sustained heating and/or resistive heating) to partially or completely disrupt the ability of a nerve to transmit a signal. Desired thermal heating effects, for example, may include raising the temperature of target neural fibers above a desired threshold to achieve non-ablative thermal alteration, or above a higher temperature to achieve ablative thermal alteration. For example, the target temperature can be above body temperature (e.g., approximately 37° C.) but less than about 45° C. for non-ablative thermal alteration, or the target temperature can be about 45° C. or higher for ablative thermal alteration. More specifically, exposure to thermal energy in excess of a body temperature of about 37° C., but below a temperature of about 45° C., may induce thermal alteration via moderate heating of target neural fibers or of vascular structures that perfuse the target fibers. In cases where vascular structures are affected, the target neural fibers may be denied perfusion resulting in necrosis of the neural tissue. For example, this may induce non-ablative thermal alteration in the fibers or structures. Exposure to heat above a temperature of about 45° C., or above about 60° C., may induce thermal alteration via substantial heating of the fibers or structures. For example, such higher temperatures may thermally ablate the target neural fibers or the vascular structures that perfuse the target fibers. In some patients, it may be desirable to achieve temperatures that thermally ablate the target neural fibers or the vascular structures, but that are less than about 90° C., or less than about 85° C., or less than about 80° C., and/or less than about 75° C. Other embodiments can include heating tissue to a variety of other suitable temperatures. Regardless of the type of heat exposure utilized to induce the thermal neuromodulation, a therapeutic effect (e.g., a reduction in renal sympathetic nerve activity (RSNA)) is expected.

Accordingly, in some embodiments, the temperature is monitored over time to ensure that sufficient temperature is applied for a sufficient treatment period to achieve treatment goals. For example, in various embodiments, heating element temperatures of 85-95° C. are targeted, although other temperatures or temperature ranges can be targeted.

In some embodiments, algorithms can be included to turn on energy to heat the element for a predetermined period, then turn off the energy and measure the rate of temperature decrease. The effect of blood flow on thermal decay can be measured and accounted for in the energy algorithm. Accordingly, blood flow and blood pressure monitors can also be included to provide information to the control algorithm controlling the delivery of energy to the heating elements.

As the example in FIG. 12 illustrates, embodiments can be implemented in which only two lead wires and two sensor wires are required to implement a direct heat modality. In various embodiments, electrodes can be included in the therapeutic assembly 21 for impedance measurement to aid in contact determination.

With direct heat applications, it is not necessary to conduct electrical current through tissue. Instead, transfer of thermal energy from the heating element to the tissue can be sufficient. Accordingly, additional features such as impedance or temperature sensors that protrude into the adventitia can be included. As described above, impedance can be used to detect contact with the vessel walls.

Where components or modules of the invention are implemented in whole or in part using software, in one embodiment, these software elements can be implemented to operate with a computing or processing module capable of carrying out the functionality described with respect thereto. One such example computing module is shown in FIG. 13. Various embodiments are described in terms of this example-computing module 200. After reading this description, it will become apparent to a person skilled in the relevant art how to implement the invention using other computing modules or architectures.

Referring now to FIG. 13, computing module 200 may represent, for example, computing or processing capabilities found within desktop, laptop and notebook computers; hand-held computing devices (PDA's, smart phones, tablets, cell phones, palmtops, etc.); mainframes, supercomputers, workstations or servers; or any other type of special-purpose or general-purpose computing devices as may be desirable or appropriate for a given application or environment. Computing module 200 might also represent computing capabilities embedded within or otherwise available to a given device. For example, a computing module might be found in other electronic devices such as, for example, digital cameras, navigation systems, cellular telephones, portable computing devices, modems, routers, WAPs, terminals and other electronic devices that might include some form of processing capability.

Computing module 200 might include, for example, one or more processors, controllers, control modules, or other processing devices, such as a processor 204. Processor 204 might be implemented using a general-purpose or special-purpose processing engine such as, for example, a microprocessor, controller, or other control logic. In the illustrated example, processor 204 is connected to a bus 202, although any communication medium can be used to facilitate interaction with other components of computing module 200 or to communicate externally.

Computing module 200 might also include one or more memory modules, simply referred to herein as main memory 208. For example, preferably random access memory (RAM) or other dynamic memory, might be used for storing information and instructions to be executed by processor 204. Main memory 208 might also be used for storing temporary variables or other intermediate information during execution of instructions to be executed by processor 204. Computing module 200 might likewise include a read only memory (“ROM”) or other static storage device coupled to bus 202 for storing static information and instructions for processor 204.

The computing module 200 might also include one or more various forms of information storage mechanism 210, which might include, for example, a media drive 212 and a storage unit interface 220. The media drive 212 might include a drive or other mechanism to support fixed or removable storage media 214. For example, a hard disk drive, a floppy disk drive, a magnetic tape drive, an optical disk drive, a CD or DVD drive (R or RW), or other removable or fixed media drive might be provided. Accordingly, storage media 214 might include, for example, a hard disk, a floppy disk, magnetic tape, cartridge, optical disk, a CD or DVD, or other fixed or removable medium that is read by, written to or accessed by media drive 212. As these examples illustrate, the storage media 214 can include a computer usable storage medium having stored therein computer software or data.

In alternative embodiments, information storage mechanism 210 might include other similar instrumentalities for allowing computer programs or other instructions or data to be loaded into computing module 200. Such instrumentalities might include, for example, a fixed or removable storage unit 222 and an interface 220. Examples of such storage units 222 and interfaces 220 can include a program cartridge and cartridge interface, a removable memory (for example, a flash memory or other removable memory module) and memory slot, a PCMCIA slot and card, and other fixed or removable storage units 222 and interfaces 220 that allow software and data to be transferred from the storage unit 222 to computing module 200.

Computing module 200 might also include a communications interface 224. Communications interface 224 might be used to allow software and data to be transferred between computing module 200 and external devices. Examples of communications interface 224 might include a modem or softmodem, a network interface (such as an Ethernet, network interface card, WiMedia, IEEE 802.XX or other interface), a communications port (such as for example, a USB port, IR port, RS232 port Bluetooth® interface, or other port), or other communications interface. Software and data transferred via communications interface 224 might typically be carried on signals, which can be electronic, electromagnetic (which includes optical) or other signals capable of being exchanged by a given communications interface 224. These signals might be provided to communications interface 224 via a channel 228. This channel 228 might carry signals and might be implemented using a wired or wireless communication medium. Some examples of a channel might include a phone line, a cellular link, an RF link, an optical link, a network interface, a local or wide area network, and other wired or wireless communications channels.

In this document, the terms “computer program medium” and “computer usable medium” are used to generally refer to media such as, for example, main memory 208, storage unit interface 220, storage media 214, and channel 228. These and other various forms of computer program media or computer usable media may be involved in carrying one or more sequences of one or more instructions to a processing device for execution. Such instructions embodied on the medium, are generally referred to as “computer program code” or a “computer program product” (which may be grouped in the form of computer programs or other groupings). When executed, such instructions might enable the computing module 200 to perform features or functions of the present disclosure as discussed herein.

### III. Pertinent Anatomy and Physiology

The following discussion provides further details regarding pertinent patient anatomy and physiology. This section is intended to supplement and expand upon the previous discussion regarding the relevant anatomy and physiology, and to provide additional context regarding the disclosed technology and the therapeutic benefits associated with renal denervation. For example, as mentioned previously, several properties of the renal vasculature may inform the design of treatment devices and associated methods for achieving renal neuromodulation via intravascular access, and impose specific design requirements for such devices. Specific design requirements may include accessing the renal artery, facilitating stable contact between the energy delivery elements of such devices and a luminal surface or wall of the renal artery, and/or effectively modulating the renal nerves with the neuromodulatory apparatus.

A. The Sympathetic Nervous System

The Sympathetic Nervous System (SNS) is a branch of the autonomic nervous system along with the enteric nervous system and parasympathetic nervous system. It is always active at a basal level (called sympathetic tone) and becomes more active during times of stress. Like other parts of the nervous system, the sympathetic nervous system operates through a series of interconnected neurons. Sympathetic neurons are frequently considered part of the peripheral nervous system (PNS), although many lie within the central nervous system (CNS). Sympathetic neurons of the spinal cord (which is part of the CNS) communicate with peripheral sympathetic neurons via a series of sympathetic ganglia. Within the ganglia, spinal cord sympathetic neurons join peripheral sympathetic neurons through synapses. Spinal cord sympathetic neurons are therefore called presynaptic (or preganglionic) neurons, while peripheral sympathetic neurons are called postsynaptic (or postganglionic) neurons.

At synapses within the sympathetic ganglia, preganglionic sympathetic neurons release acetylcholine, a chemical messenger that binds and activates nicotinic acetylcholine receptors on postganglionic neurons. In response to this stimulus, postganglionic neurons principally release noradrenaline (norepinephrine). Prolonged activation may elicit the release of adrenaline from the adrenal medulla.

Once released, norepinephrine and epinephrine bind adrenergic receptors on peripheral tissues. Binding to adrenergic receptors causes a neuronal and hormonal response. The physiologic manifestations include pupil dilation, increased heart rate, occasional vomiting, and increased blood pressure. Increased sweating is also seen due to binding of cholinergic receptors of the sweat glands.

The sympathetic nervous system is responsible for up- and down-regulating many homeostatic mechanisms in living organisms. Fibers from the SNS innervate tissues in almost every organ system, providing at least some regulatory function to things as diverse as pupil diameter, gut motility, and urinary output. This response is also known as sympatho-adrenal response of the body, as the preganglionic sympathetic fibers that end in the adrenal medulla (but also all other sympathetic fibers) secrete acetylcholine, which activates the secretion of adrenaline (epinephrine) and to a lesser extent noradrenaline (norepinephrine). Therefore, this response that acts primarily on the cardiovascular system is mediated directly via impulses transmitted through the sympathetic nervous system and indirectly via catecholamines secreted from the adrenal medulla.

Science typically looks at the SNS as an automatic regulation system, that is, one that operates without the intervention of conscious thought. Some evolutionary theorists suggest that the sympathetic nervous system operated in early organisms to maintain survival as the sympathetic nervous system is responsible for priming the body for action. One example of this priming is in the moments before waking, in which sympathetic outflow spontaneously increases in preparation for action.

1. The Sympathetic Chain

As shown in FIG. 14, the SNS provides a network of nerves that allows the brain to communicate with the body. Sympathetic nerves originate inside the vertebral column, toward the middle of the spinal cord in the intermediolateral cell column (or lateral horn), beginning at the first thoracic segment of the spinal cord and are thought to extend to the second or third lumbar segments. Because its cells begin in the thoracic and lumbar regions of the spinal cord, the SNS is said to have a thoracolumbar outflow. Axons of these nerves leave the spinal cord through the anterior rootlet/root. They pass near the spinal (sensory) ganglion, where they enter the anterior rami of the spinal nerves. However, unlike somatic innervation, they quickly separate out through white rami connectors which connect to either the paravertebral (which lie near the vertebral column) or prevertebral (which lie near the aortic bifurcation) ganglia extending alongside the spinal column.

In order to reach the target organs and glands, the axons should travel long distances in the body, and, to accomplish this, many axons relay their message to a second cell through synaptic transmission. The ends of the axons link across a space, the synapse, to the dendrites of the second cell. The first cell (the presynaptic cell) sends a neurotransmitter across the synaptic cleft where it activates the second cell (the postsynaptic cell). The message is then carried to the final destination.

In the SNS and other components of the peripheral nervous system, these synapses are made at sites called ganglia. The cell that sends its fiber is called a preganglionic cell, while the cell whose fiber leaves the ganglion is called a postganglionic cell. As mentioned previously, the preganglionic cells of the SNS are located between the first thoracic (T1) segment and third lumbar (L3) segments of the spinal cord. Postganglionic cells have their cell bodies in the ganglia and send their axons to target organs or glands.

The ganglia include not just the sympathetic trunks but also the cervical ganglia (superior, middle and inferior), which sends sympathetic nerve fibers to the head and thorax organs, and the celiac and mesenteric ganglia (which send sympathetic fibers to the gut).

2. Innervation of the Kidneys

As shown in FIG. 15, the kidney is innervated by the renal plexus (RP), which is intimately associated with the renal artery. The renal plexus (RP) is an autonomic plexus that surrounds the renal artery and is embedded within the adventitia of the renal artery. The renal plexus (RP) extends along the renal artery until it arrives at the substance of the kidney. Fibers contributing to the renal plexus (RP) arise from the celiac ganglion, the superior mesenteric ganglion, the aorticorenal ganglion and the aortic plexus. The renal plexus (RP), also referred to as the renal nerve, is predominantly comprised of sympathetic components. There is no (or at least very minimal) parasympathetic innervation of the kidney.

Preganglionic neuronal cell bodies are located in the intermediolateral cell column of the spinal cord. Preganglionic axons pass through the paravertebral ganglia (they do not synapse) to become the lesser splanchnic nerve, the least splanchnic nerve, first lumbar splanchnic nerve, second lumbar splanchnic nerve, and travel to the celiac ganglion, the superior mesenteric ganglion, and the aorticorenal ganglion. Postganglionic neuronal cell bodies exit the celiac ganglion, the superior mesenteric ganglion, and the aorticorenal ganglion to the renal plexus (RP) and are distributed to the renal vasculature.

3. Renal Sympathetic Neural Activity

Messages travel through the SNS in a bidirectional flow. Efferent messages may trigger changes in different parts of the body simultaneously. For example, the sympathetic nervous system may accelerate heart rate; widen bronchial passages; decrease motility (movement) of the large intestine; constrict blood vessels; increase peristalsis in the esophagus; cause pupil dilation, piloerection (goose bumps) and perspiration (sweating); and raise blood pressure. Afferent messages carry signals from various organs and sensory receptors in the body to other organs and, particularly, the brain.

Hypertension, heart failure and chronic kidney disease are a few of many disease states that result from chronic activation of the SNS, especially the renal sympathetic nervous system. Chronic activation of the SNS is a maladaptive response that drives the progression of these disease states. Pharmaceutical management of the renin-angiotensin-aldosterone system (RAAS) has been a longstanding, but somewhat ineffective, approach for reducing over-activity of the SNS.

As mentioned above, the renal sympathetic nervous system has been identified as a major contributor to the complex pathophysiology of hypertension, states of volume overload (such as heart failure), and progressive renal disease, both experimentally and in humans. Studies employing radiotracer dilution methodology to measure overflow of norepinephrine from the kidneys to plasma revealed increased renal norepinephrine (NE) spillover rates in patients with essential hypertension, particularly so in young hypertensive subjects, which in concert with increased NE spillover from the heart, is consistent with the hemodynamic profile typically seen in early hypertension and characterized by an increased heart rate, cardiac output, and renovascular resistance. It is now known that essential hypertension is commonly neurogenic, often accompanied by pronounced sympathetic nervous system overactivity.

Activation of cardiorenal sympathetic nerve activity is even more pronounced in heart failure, as demonstrated by an exaggerated increase of NE overflow from the heart and the kidneys to plasma in this patient group. In line with this notion is the recent demonstration of a strong negative predictive value of renal sympathetic activation on all-cause mortality and heart transplantation in patients with congestive heart failure, which is independent of overall sympathetic activity, glomerular filtration rate, and left ventricular ejection fraction. These findings support the notion that treatment regimens that are designed to reduce renal sympathetic stimulation have the potential to improve survival in patients with heart failure.

Both chronic and end stage renal disease are characterized by heightened sympathetic nervous activation. In patients with end stage renal disease, plasma levels of norepinephrine above the median have been demonstrated to be predictive for both all-cause death and death from cardiovascular disease. This is also true for patients suffering from diabetic or contrast nephropathy. There is compelling evidence suggesting that sensory afferent signals originating from the diseased kidneys are major contributors to initiating and sustaining elevated central sympathetic outflow in this patient group; this facilitates the occurrence of the well-known adverse consequences of chronic sympathetic over activity, such as hypertension, left ventricular hypertrophy, ventricular arrhythmias, sudden cardiac death, insulin resistance, diabetes, and metabolic syndrome.

i. Renal Sympathetic Efferent Activity

Sympathetic nerves to the kidneys terminate in the blood vessels, the juxtaglomerular apparatus and the renal tubules. Stimulation of the renal sympathetic nerves causes increased renin release, increased sodium (Na⁺) reabsorption, and a reduction of renal blood flow. These components of the neural regulation of renal function are considerably stimulated in disease states characterized by heightened sympathetic tone and clearly contribute to the rise in blood pressure in hypertensive patients. The reduction of renal blood flow and glomerular filtration rate as a result of renal sympathetic efferent stimulation is likely a cornerstone of the loss of renal function in cardio-renal syndrome, which is renal dysfunction as a progressive complication of chronic heart failure, with a clinical course that typically fluctuates with the patient's clinical status and treatment. Pharmacologic strategies to thwart the consequences of renal efferent sympathetic stimulation include centrally acting sympatholytic drugs, beta blockers (intended to reduce renin release), angiotensin converting enzyme inhibitors and receptor blockers (intended to block the action of angiotensin II and aldosterone activation consequent to renin release) and diuretics (intended to counter the renal sympathetic mediated sodium and water retention). However, the current pharmacologic strategies have significant limitations including limited efficacy, compliance issues, side effects and others.

ii. Renal Sensory Afferent Nerve Activity

The kidneys communicate with integral structures in the central nervous system via renal sensory afferent nerves. Several forms of “renal injury” may induce activation of sensory afferent signals. For example, renal ischemia, reduction in stroke volume or renal blood flow, or an abundance of adenosine enzyme may trigger activation of afferent neural communication. As shown in FIGS. 16a and 16b, this afferent communication might be from the kidney to the brain or might be from one kidney to the other kidney (via the central nervous system). These afferent signals are centrally integrated and may result in increased sympathetic outflow. This sympathetic drive is directed towards the kidneys, thereby activating the RAAS and inducing increased renin secretion, sodium retention, volume retention and vasoconstriction. Central sympathetic over activity also impacts other organs and bodily structures innervated by sympathetic nerves such as the heart and the peripheral vasculature, resulting in the described adverse effects of sympathetic activation, several aspects of which also contribute to the rise in blood pressure.

The physiology therefore suggests that (i) modulation of tissue with efferent sympathetic nerves will reduce inappropriate renin release, salt retention, and reduction of renal blood flow, and that (ii) modulation of tissue with afferent sensory nerves will reduce the systemic contribution to hypertension and other disease states associated with increased central sympathetic tone through its direct effect on the posterior hypothalamus as well as the contralateral kidney. In addition to the central hypotensive effects of afferent renal denervation, a desirable reduction of central sympathetic outflow to various other sympathetically innervated organs such as the heart and the vasculature is anticipated.

B. Additional Clinical Benefits of Renal Denervation

As provided above, renal denervation is likely to be valuable in the treatment of several clinical conditions characterized by increased overall and particularly renal sympathetic activity such as hypertension, metabolic syndrome, insulin resistance, diabetes, left ventricular hypertrophy, chronic end stage renal disease, inappropriate fluid retention in heart failure, cardio-renal syndrome, osteoporosis and sudden death. Since the reduction of afferent neural signals contributes to the systemic reduction of sympathetic tone/drive, renal denervation might also be useful in treating other conditions associated with systemic sympathetic hyperactivity. Accordingly, renal denervation may also benefit other organs and bodily structures innervated by sympathetic nerves. For example, as previously discussed, a reduction in central sympathetic drive may reduce the insulin resistance that afflicts people with metabolic syndrome and Type II diabetics.

C. Achieving Intravascular Access to the Renal Artery

In accordance with the present technology, neuromodulation of a left and/or right renal plexus (RP), which is intimately associated with a left and/or right renal artery, may be achieved through intravascular access. As FIG. 17a shows, blood moved by contractions of the heart is conveyed from the left ventricle of the heart by the aorta. The aorta descends through the thorax and branches into the left and right renal arteries. Below the renal arteries, the aorta bifurcates at the left and right iliac arteries. The left and right iliac arteries descend, respectively, through the left and right legs and join the left and right femoral arteries.

As FIG. 17b shows, the blood collects in veins and returns to the heart, through the femoral veins into the iliac veins and into the inferior vena cava. The inferior vena cava branches into the left and right renal veins. Above the renal veins, the inferior vena cava ascends to convey blood into the right atrium of the heart. From the right atrium, the blood is pumped through the right ventricle into the lungs, where it is oxygenated. From the lungs, the oxygenated blood is conveyed into the left atrium. From the left atrium, the oxygenated blood is conveyed by the left ventricle back to the aorta.

As will be described in greater detail later, the femoral artery may be accessed and cannulated at the base of the femoral triangle just inferior to the midpoint of the inguinal ligament. A catheter may be inserted percutaneously into the femoral artery through this access site, passed through the iliac artery and aorta, and placed into either the left or right renal artery. This comprises an intravascular path that offers minimally invasive access to a respective renal artery and/or other renal blood vessels.

The wrist, upper arm, and shoulder region provide other locations for introduction of catheters into the arterial system. For example, catheterization of either the radial, brachial, or axillary artery may be utilized in select cases. Catheters introduced via these access points may be passed through the subclavian artery on the left side (or via the subclavian and brachiocephalic arteries on the right side), through the aortic arch, down the descending aorta and into the renal arteries using standard angiographic technique.

D. Properties and Characteristics of the Renal Vasculature

Since neuromodulation of a left and/or right renal plexus (RP) may be achieved in accordance with the present technology through intravascular access, properties and characteristics of the renal vasculature may impose constraints upon and/or inform the design of apparatus, systems, and methods for achieving such renal neuromodulation. Some of these properties and characteristics may vary across the patient population and/or within a specific patient across time, as well as in response to disease states, such as hypertension, chronic kidney disease, vascular disease, end-stage renal disease, insulin resistance, diabetes, metabolic syndrome, etc. These properties and characteristics, as explained herein, may have bearing on the efficacy of the procedure and the specific design of the intravascular device. Properties of interest may include, for example, material/mechanical, spatial, fluid dynamic/hemodynamic and/or thermodynamic properties.

As discussed previously, a catheter may be advanced percutaneously into either the left or right renal artery via a minimally invasive intravascular path. However, minimally invasive renal arterial access may be challenging, for example, because as compared to some other arteries that are routinely accessed using catheters, the renal arteries are often extremely tortuous, may be of relatively small diameter, and/or may be of relatively short length. Furthermore, renal arterial atherosclerosis is common in many patients, particularly those with cardiovascular disease. Renal arterial anatomy also may vary significantly from patient to patient, which further complicates minimally invasive access. Significant inter-patient variation may be seen, for example, in relative tortuosity, diameter, length, and/or atherosclerotic plaque burden, as well as in the take-off angle at which a renal artery branches from the aorta. Apparatus, systems and methods for achieving renal neuromodulation via intravascular access should account for these and other aspects of renal arterial anatomy and its variation across the patient population when minimally invasively accessing a renal artery.

In addition to complicating renal arterial access, specifics of the renal anatomy also complicate establishment of stable contact between neuromodulatory apparatus and a luminal surface or wall of a renal artery. When the neuromodulatory apparatus includes an energy delivery element, such as an electrode, consistent positioning and appropriate contact force applied by the energy delivery element to the vessel wall are important for predictability. However, navigation is impeded by the tight space within a renal artery, as well as tortuosity of the artery. Furthermore, establishing consistent contact is complicated by patient movement, respiration, and/or the cardiac cycle because these factors may cause significant movement of the renal artery relative to the aorta, and the cardiac cycle may transiently distend the renal artery (i.e., cause the wall of the artery to pulse).

Even after accessing a renal artery and facilitating stable contact between neuromodulatory apparatus and a luminal surface of the artery, nerves in and around the adventia of the artery should be safely modulated via the neuromodulatory apparatus. Effectively applying thermal treatment from within a renal artery is non-trivial given the potential clinical complications associated with such treatment. For example, the intima and media of the renal artery are highly vulnerable to thermal injury. As discussed in greater detail below, the intima-media thickness separating the vessel lumen from its adventitia means that target renal nerves may be multiple millimeters distant from the luminal surface of the artery. Sufficient energy should be delivered to or heat removed from the target renal nerves to modulate the target renal nerves without excessively cooling or heating the vessel wall to the extent that the wall is frozen, desiccated, or otherwise potentially affected to an undesirable extent. A potential clinical complication associated with excessive heating is thrombus formation from coagulating blood flowing through the artery. Given that this thrombus may cause a kidney infarct, thereby causing irreversible damage to the kidney, thermal treatment from within the renal artery should be applied carefully. Accordingly, the complex fluid mechanics and thermodynamic conditions present in the renal artery during treatment, particularly those that may impact heat transfer dynamics at the treatment site, may be important in applying energy (e.g., heating thermal energy) and/or removing heat from the tissue (e.g., cooling thermal conditions) from within the renal artery.

The neuromodulatory apparatus should also be configured to allow for adjustable positioning and repositioning of the energy delivery element within the renal artery since location of treatment may also impact clinical efficacy. For example, it may be tempting to apply a full circumferential treatment from within the renal artery given that the renal nerves may be spaced circumferentially around a renal artery. In some situations, full-circle lesion likely resulting from a continuous circumferential treatment may be potentially related to renal artery stenosis. Therefore, the formation of more complex lesions along a longitudinal dimension of the renal artery via the mesh structures described herein and/or repositioning of the neuromodulatory apparatus to multiple treatment locations may be desirable. It should be noted, however, that a benefit of creating a circumferential ablation may outweigh the potential of renal artery stenosis or the risk may be mitigated with certain embodiments or in certain patients and creating a circumferential ablation could be a goal. Additionally, variable positioning and repositioning of the neuromodulatory apparatus may prove to be useful in circumstances where the renal artery is particularly tortuous or where there are proximal branch vessels off the renal artery main vessel, making treatment in certain locations challenging. Manipulation of a device in a renal artery should also consider mechanical injury imposed by the device on the renal artery. Motion of a device in an artery, for example by inserting, manipulating, negotiating bends and so forth, may contribute to dissection, perforation, denuding intima, or disrupting the interior elastic lamina.

Blood flow through a renal artery may be temporarily occluded for a short time with minimal or no complications. However, occlusion for a significant amount of time should be avoided to prevent injury to the kidney such as ischemia. It could be beneficial to avoid occlusion all together or, if occlusion is beneficial to the embodiment, to limit the duration of occlusion, for example to 2-5 minutes.

Based on the above described challenges of (1) renal artery intervention, (2) consistent and stable placement of the treatment element against the vessel wall, (3) effective application of treatment across the vessel wall, (4) positioning and potentially repositioning the treatment apparatus to allow for multiple treatment locations, and (5) avoiding or limiting duration of blood flow occlusion, various independent and dependent properties of the renal vasculature that may be of interest include, for example, (a) vessel diameter, vessel length, intima-media thickness, coefficient of friction, and tortuosity; (b) distensibility, stiffness and modulus of elasticity of the vessel wall; (c) peak systolic, end-diastolic blood flow velocity, as well as the mean systolic-diastolic peak blood flow velocity, and mean/max volumetric blood flow rate; (d) specific heat capacity of blood and/or of the vessel wall, thermal conductivity of blood and/or of the vessel wall, and/or thermal convectivity of blood flow past a vessel wall treatment site and/or radiative heat transfer; (e) renal artery motion relative to the aorta induced by respiration, patient movement, and/or blood flow pulsatility; and (f) the take-off angle of a renal artery relative to the aorta. These properties will be discussed in greater detail with respect to the renal arteries. However, dependent on the apparatus, systems and methods utilized to achieve renal neuromodulation, such properties of the renal arteries, also may guide and/or constrain design characteristics.

As noted above, an apparatus positioned within a renal artery should conform to the geometry of the artery. Renal artery vessel diameter, DRA, typically is in a range of about 2-10 mm, with most of the patient population having a DRA of about 4 mm to about 8 mm and an average of about 6 mm. Renal artery vessel length, LRA, between its ostium at the aorta/renal artery juncture and its distal branchings, generally is in a range of about 5-70 mm, and a significant portion of the patient population is in a range of about 20-50 mm. Since the target renal plexus is embedded within the adventitia of the renal artery, the composite Intima-Media Thickness, IMT, (i.e., the radial outward distance from the artery's luminal surface to the adventitia containing target neural structures) also is notable and generally is in a range of about 0.5-2.5 mm, with an average of about 1.5 mm. Although a certain depth of treatment is important to reach the target neural fibers, the treatment should not be too deep (e.g., >5 mm from inner wall of the renal artery) to avoid non-target tissue and anatomical structures such as the renal vein.

An additional property of the renal artery that may be of interest is the degree of renal motion relative to the aorta, induced by respiration and/or blood flow pulsatility. A patient's kidney, which located at the distal end of the renal artery, may move as much as 4″ cranially with respiratory excursion. This may impart significant motion to the renal artery connecting the aorta and the kidney, thereby requiring from the neuromodulatory apparatus a unique balance of stiffness and flexibility to maintain contact between the thermal treatment element and the vessel wall during cycles of respiration. Furthermore, the take-off angle between the renal artery and the aorta may vary significantly between patients, and also may vary dynamically within a patient, e.g., due to kidney motion. The take-off angle generally may be in a range of about 30 degrees-135 degrees.

While various embodiments of the present invention have been described above, it should be understood that they have been presented by way of example only, and not of limitation. Likewise, the various diagrams may depict an example architectural or other configuration for the invention, which is done to aid in understanding the features and functionality that can be included in the invention. The invention is not restricted to the illustrated example architectures or configurations, but the desired features can be implemented using a variety of alternative architectures and configurations. Indeed, it will be apparent to one of skill in the art how alternative functional, logical or physical partitioning and configurations can be implemented to implement the desired features of the present invention. Also, a multitude of different constituent module names other than those depicted herein can be applied to the various partitions. Additionally, with regard to flow diagrams, operational descriptions and method claims, the order in which the steps are presented herein shall not mandate that various embodiments be implemented to perform the recited functionality in the same order unless the context dictates otherwise.

Although the invention is described above in terms of various exemplary embodiments and implementations, it should be understood that the various features, aspects and functionality described in one or more of the individual embodiments are not limited in their applicability to the particular embodiment with which they are described, but instead can be applied, alone or in various combinations, to one or more of the other embodiments of the invention, whether or not such embodiments are described and whether or not such features are presented as being a part of a described embodiment. Thus, the breadth and scope of the present invention should not be limited by any of the above-described exemplary embodiments.

Terms and phrases used in this document, and variations thereof, unless otherwise expressly stated, should be construed as open ended as opposed to limiting. As examples of the foregoing: the term “including” should be read as meaning “including, without limitation” or the like; the term “example” is used to provide exemplary instances of the item in discussion, not an exhaustive or limiting list thereof; the terms “a” or “an” should be read as meaning “at least one,” “one or more” or the like; and adjectives such as “conventional,” “traditional,” “normal,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Likewise, where this document refers to technologies that would be apparent or known to one of ordinary skill in the art, such technologies encompass those apparent or known to the skilled artisan now or at any time in the future.

The presence of broadening words and phrases such as “one or more,” “at least,” “but not limited to” or other like phrases in some instances shall not be read to mean that the narrower case is intended or required in instances where such broadening phrases may be absent. The use of the term “module” does not imply that the components or functionality described or claimed as part of the module are all configured in a common package. Indeed, any or all of the various components of a module, whether control logic or other components, can be combined in a single package or separately maintained and can further be distributed in multiple groupings or packages or across multiple locations.

Additionally, the various embodiments set forth herein are described in terms of exemplary block diagrams, flow charts and other illustrations. As will become apparent to one of ordinary skill in the art after reading this document, the illustrated embodiments and their various alternatives can be implemented without confinement to the illustrated examples. For example, block diagrams and their accompanying description should not be construed as mandating a particular architecture or configuration.

1-18. (canceled)

19. An apparatus for neuromodulation treatment, comprising a therapeutic assembly configured to be delivered to a treatment site within a vessel;
wherein the therapeutic assembly comprises a heating element configured to be positioned in contact with a vessel wall to deliver heat to the vessel wall.

20-21. (canceled)

22. The apparatus according to claim 19, wherein the therapeutic assembly further comprises a core element having a length, and the heating element is disposed on all or substantially all of the length of the core element.

23. The apparatus according to claim 22, wherein the heating element comprises wire wrapped around all or substantially all of the length of the core element.

24. The apparatus according to claim 19, wherein the therapeutic assembly further comprises an inflatable balloon, and the heating element comprises a resistive heating element disposed on the inflatable balloon.

25. The apparatus according to claim 23, wherein the heating element comprises a resistive wire wrapped around a surface of the balloon.

26. The apparatus according to claim 25, further comprising a temperature sensor configured to measure a temperature of the heating element.

27. The apparatus according to claim 23, wherein the heating element comprises a conductive pattern printed on a surface of the balloon.

28. The apparatus according to claim 23, further comprising a temperature sensor configured to measure a temperature of the heating element.

29-30. (canceled)

31. The apparatus according to claim 19, wherein the heating element is a direct heat heating element.

32. A method for neuromodulation through the application of heat, comprising: positioning a therapeutic assembly within a blood vessel of a patient, the therapeutic assembly comprising a heating element; generating heat from the heating element; and neuromodulating innervated tissue of a wall of the blood vessel via the application of the heat generated by the heating element.

33. The method of claim 32, further comprising positioning the therapeutic assembly within the blood vessel in a first state and expanding the therapeutic assembly to a second state such that the heating element comes into contact with an inner surface of the wall of the blood vessel.

34. (canceled)

35. The method of claim 32, wherein the heating element is a resistive heating element and generating heat from the heating element comprises applying an electrical current to the resistive heating element.

36. (canceled)

37. The method of claim 35, further comprising measuring a temperature of the resistive heating element during treatment and adjusting the current applied to the resistive heating element to achieve a desired temperature.

38. The method of claim 32, wherein neuromodulating the tissue comprises at least partially denervating the tissue.

39. The method of claim 32, further comprising maintaining the heating element within a predetermined temperature range for a period of time.

40. The method of claim 39, wherein the period of time is determined based on the temperature range and the amount of neuromodulation desired.

41. (canceled)

42. The method of claim 32, wherein generating heat from the heating element comprises delivering energy to the heating element to increase its temperature to a determined temperature or to a temperature within a determined range of temperatures.

43. (canceled)

44. The method of claim 42, further comprising measuring an amount of time at which the heating element is at the determined temperature or within the temperature range, and ceasing energy delivery when the amount of time reaches a determined amount.

45. The method of claim 42, further comprising determining a time period over which the neuromodulation is to be performed, and terminating the energy delivery when the heating element has been at the determined temperature or temperature range for the determined period of time.

46. (canceled)

47. The method of claim 42, further comprising reducing or ceasing energy delivery if the temperature of the heating element reaches or exceeds a predetermined threshold.
